(19) United States
(12) Patent Application Publication
(10) Pub. No.: US 2009/0030295 A1
(43) Pub. Date: Jan. 29, 2009

(54) INSTRUMENT FOR MEASURING CONCENTRATION OF LIVING BODY INGREDIENT

(76) Inventors: Masahiko Shioi, Osaka (JP); Shinji Uchida, Osaka (JP); Yoshiko Miyamoto, Osaka (JP)

Correspondence Address: MARK D. SARALINO (PAN);RENNER, OTTO, BOISSELLE & SKLAR, LLP, 1621 EUCLID AVENUE, 19TH FLOOR, CLEVELAND, OH, 44115, US

(21) Appl. No.: 12/281,809

(22) PCT Filed: Mar. 8, 2007
(86) PCT No.: PCT/JP2007/054554

§ 371 Date: Sep. 5, 2008

(30) Foreign Application Priority Data
Mar. 10, 2006 (JP) 2006-065361

(51) Int. Cl. A61B 5/1455

(52) U.S. Cl. 600/316

(57) ABSTRACT

A biological constituent concentration measuring device that can measure a biological constituent concentration highly accurately using a radiation that has come from an eardrum is provided.

A measuring device for measuring concentration of a biological constituent includes: an image capturing section for capturing an image of an eardrum; a processing section for generating tilt information concerning tilt of the eardrum based on a first image capturing information obtained by capturing an image of a first area of the eardrum and a second image capturing information obtained by capturing an image of a second area of the eardrum, which is different from the first area; an infrared sensor for sensing infrared radiation that has been radiated from the eardrum; and a computing section for calculating the concentration of a biological constituent based on the infrared radiation sensed and the tilt information.

### TECHNICAL FIELD

The present invention relates to a device for measuring a biological constituent concentration such as a glucose concentration non-invasively without collecting blood, for example.

### BACKGROUND ART

A non-invasive blood glucose meter for calculating a glucose level by measuring the intensity of radiation that has come from an eardrum has been proposed as a conventional biological information measuring device. For example, Patent Document No. 1 discloses a non-invasive blood glucose meter, which includes a mirror that is small enough to be introduced into an ear canal and which irradiates the eardrum with the near infrared radiation or a thermal radiation by way of that mirror and detects the light that has been reflected from the eardrum, thereby calculating the glucose level based on the result of the detection. Also, Patent Document No. 2 discloses a non-invasive blood glucose meter, which includes a probe to be inserted into an acoustic foramen and which detects the infrared radiation that has been produced from the inner ear and radiated from the eardrum at the probe with the eardrum and ear canal cooled and then subjects the detected infrared radiation to a spectral analysis, thereby obtaining the glucose level. Furthermore, Patent Document No. 3 discloses a non-invasive blood glucose meter, which includes a reflective mirror to be inserted into an acoustic foramen and which detects the radiation that has come from the eardrum using the reflective mirror and subjects the radiation detected to a spectral analysis, thereby obtaining the glucose level.
Patent Document No. 1: description and drawings of U.S. Pat. No. 5,115,133
Patent Document No. 2: description and drawings of U.S. Pat. No. 6,002,953
Patent Document No. 3: description and drawings of U.S. Pat. No. 5,666,956

### DISCLOSURE OF INVENTION

### Problems to be Solved by the Invention

However, it is known that the angle formed by the eardrum with respect to a plane that intersects at right angles with an axis connecting the center of the entrance of the ear canal to the navel of the tympanic membrane (or eardrum) varies from one person to another. Also, the mirror or probe being inserted into the acoustic foramen may also have a varied insertion angle, thus possibly changing the positions of their end face with respect to the eardrum every time it is inserted. The degree of tilt of the eardrum with respect to the end face of the mirror or probe inserted into the acoustic foramen has an influence on the quantity of radiation that has come from the eardrum and incident on the mirror or probe. Thus, the conventional non-invasive blood glucose meters cannot measure the biological constituent concentration consistently, which is a problem.

In order to overcome the problems described above, the present invention has an object of providing a biological constituent concentration measuring device that can measure a biological constituent concentration highly accurately using the radiation that has come from the eardrum.

### Means for Solving the Problems

A measuring device for measuring concentration of a biological constituent according to the present invention includes: an image capturing section for capturing an image of an eardrum; a processing section for generating tilt information concerning tilt of the eardrum based on a first image capturing information obtained by capturing an image of a first area of the eardrum and a second image capturing information obtained by capturing an image of a second area of the eardrum, which is different from the first area; an infrared sensor for sensing infrared radiation that has been radiated from the eardrum; and a computing section for calculating concentration of a biological constituent based on the infrared radiation sensed and the tilt information.

The image capturing section may include an imaging device that has multiple pixels. The processing section may generate the tilt information by using the output of one of the multiple pixels that is associated with an imaging point in the first area as the first image capturing information and using the output of another one of the multiple pixels that is associated with an imaging point in the second area as the second image capturing information.

The image capturing section may further include: a light source that emits light; a lens for condensing the light, which has been emitted and then reflected from an acoustic foramen, onto the imaging device; an actuator for driving the lens; an actuator control section for controlling the actuator; and an extracting section for extracting the output of one of the pixels that is associated with an in-focus area based on the image capturing information that has been obtained by the imaging device. The extracting section may extract, as the first image capturing information, the output of at least one first pixel that is associated with the first area in which the light is focused when the lens is located at a first position and the extracting section may also extract, as the second image capturing information, the output of at least one second pixel that is associated with the second area in which the light is focused when the lens is located at a second position. The processing section may calculate an interval between the first and second pixels based on the first image capturing information and the second image capturing information. And the computing section may calculate the concentration of the biological constituent based on the interval and the infrared radiation sensed.

The processing section may calculate a distance that the lens has gone when reaching the second position from the first position, and the computing section may calculate the concentration of the biological constituent further based on the distance.

The measuring device may further include: a detecting section for detecting an image portion corresponding to the eardrum based on the image capturing information that has been provided as an image from the image capturing section, and an optical path control element for controlling the optical path of the infrared radiation that has been radiated from the eardrum based on the image portion detected such that the infrared radiation is selectively incident on one of the multiple pixels, associated with the image portion, on the imaging device.

The measuring device may further include a waveguide to be inserted into the acoustic foramen. The waveguide may output to the acoustic foramen the light that has been emitted from the light source and may receive the light that has been reflected from the acoustic foramen and the infrared radiation that has been radiated from the eardrum.

The measuring device may further include infrared radiation source for increasing intensity of the infrared radiation that has been radiated from the eardrum, and the detecting section may output a signal representing the intensity of the infrared radiation received.

The measuring device may further include an output section for outputting information concerning the calculated concentration of the biological constituent.

The output section may output the information about the biological constituent concentration to a display.

### EFFECTS OF THE INVENTION

A biological constituent concentration measuring device according to the present invention captures images of first and second areas of the eardrum, thereby obtaining first and second pieces of image capturing information. Since the eardrum is tilted, imaging points (or focal lengths) are different when the images of the first and second areas are captured. That is why information about the tilt angle of the eardrum can be obtained based on the focal lengths and the first and second pieces of image capturing information. And the concentration of the biological constituent is calculated using the infrared radiation radiated from the eardrum and the tilt information about the tilt angle of the eardrum. The concentration of the biological constituent can be measured highly precisely because the concentration is calculated based on the intensity of the infrared radiation radiated from the eardrum and with the tilt angle of the eardrum taken into account. The interval between the imaging points when images of the first and second areas are captured may be either fixed or measured every time.

### BRIEF DESCRIPTION OF DRAWINGS

FIG. 1 is a perspective view illustrating the appearance of a biological constituent concentration measuring device 100 as a first specific preferred embodiment of the present invention.

FIG. 2 shows the hardware configuration of the measuring device 100.

FIG. 3 is a perspective view illustrating an optical filter wheel 106.

FIG. 4 illustrates an image of an acoustic foramen 200 that has been shot with an imaging device 148.

FIG. 5 illustrates an image of an eardrum 202 that has been shot with the imaging device 148 when a condenser lens 146 is located at a first position.

FIG. 6 illustrates an image of the eardrum 202 that has been shot with the imaging device 148 when the condenser lens 146 is located at a second position.

FIG. 7 shows the group of linearly arranged pixels A (pixels in line A) when the condenser lens 146 is located at the first position and the group of linearly arranged pixels B (pixels in line B) when the condenser lens 146 is located at the second position.

FIG. 8 is a cross-sectional view showing where the waveguide 104 that has been inserted into the acoustic foramen 200 is located with respect to the eardrum 202.

FIG. 9 is a perspective view illustrating the appearance of a biological constituent concentration measuring device 300 according to a second specific preferred embodiment of the present invention.

FIG. 10 shows the configuration of the biological constituent concentration measuring device 300 of the second preferred embodiment.

### DESCRIPTION OF REFERENCE NUMERALS

100, 300 biological constituent concentration measuring device
101 power switch
102 body
103 measuring start switch
104 waveguide
106 optical filter wheel
108 infrared sensor
110 microcomputer
112 memory
114 display
116 power supply
118 chopper
120 liquid crystal shutter
1252 first optical filter
123 ring
124 second optical filter
125 shaft
126 sensing area
130 pre-amplifier
132 band-pass filter
134 synchronous demodulator
136 low pass filter
138 A/D converter
140 light source
142 first half mirror
144 second half mirror
146 condenser lens
148 imaging device
150 actuator
152 lens frame
154 position sensor
156 timer
158 buzzer
200 acoustic foramen
202 eardrum
204 ear canal
501 pixel
502, 502a, 502b, 602, 602a, 602b in-focus pixel
503 out-of-focus pixel
700 infrared radiation source
702 third half mirror

### BEST MODE FOR CARRYING OUT THE INVENTION

If the infrared radiation coming from an organism is measured, information about a biological constituent concentration such as a blood glucose level can be obtained. Hereinafter, that principle will be illustrated first, and then first and second specific preferred embodiments of a biological constituent concentration measuring device according to the present invention will be set forth.

The radiation energy W of the infrared radiation that has been emitted as thermal radiation from an organism is represented by the following Equations (1) and (2):

$$\begin{matrix}W = S {\int }_{{λ}_{1}}^{{λ}_{2}} ɛ ( λ ) \cdot {W}_{0} ( T , λ ) d λ ( W ) & ( 1 ) \\ {W}_{0} ( λ , T ) = 2 {hc}^{2} {\{ {λ}^{5} \cdot [ exp ( hc / λ kT ) - 1 ] \}}^{- 1} ( W \text{/} {cm}^{2} \cdot μ m ) & ( 2 )\end{matrix}$$

where W is the radiation energy of the infrared radiation that has been emitted as thermal radiation from an organism, ε (λ) is the emissivity of the organism at a wavelength λ, W₀ (λ, T) is the spectral radiant density of a thermal radiation from the blackbody at the wavelength λ and a temperature T, h is Planck's constant (where h=6.625×10⁻³⁴ W·S²), c is the velocity of light (where c 2.998×10¹⁰ cm/s), λ₁ and λ₂ are wavelengths (μm) of infrared radiations emitted as thermal radiations from the organism, T is the temperature (K) of the organism, S is the detection area (cm²) and k is Boltzmann constant.

According to Equation (1), if the detection area S is constant, the radiation energy W of the infrared radiation emitted as a thermal radiation from an organism depends on the emissivity ε (λ) of the organism at a wavelength λ. According to the Kirchhoff's law on radiation, the emissivity and the absorptivity are equal to each other at the same temperature and at the same wavelength.

ε(λ)=α(λ) (3)

where α (λ) is the absorptivity of the organism at the wavelength λ.

That is why it can be seen that when the emissivity needs to be obtained, the absorptivity may be calculated. Based on the principle of energy conservation, the absorptivity, the transmittance and the reflectance satisfy the following Equation (4):

α(λ)+r(λ)+t(λ)=1 (4)

where r (λ) is the reflectance of the organism at the wavelength λ and t (λ) is the transmittance of the organism at the wavelength λ.

Therefore, the emissivity can be calculated by the following Equation (5) using the transmittance and the reflectance:

ε(λ)=α(λ)=1−r(λ)−t(λ) (5)

The transmittance is represented as the ratio of the intensity of the light that has been transmitted through an object of interest to that of the incoming light. The intensity of the incoming light and that of the light that has been transmitted through the object of interest are given by the Lambert-Beer law:

$${I}_{t} ( λ ) = {I}_{0} ( λ ) exp ( - \frac{4 π k ( λ )}{λ} d ) \qquad ( 6 )$$

where Iₜ is the intensity of the transmitted light, I₀ is the intensity of the incoming light, d is the thickness of the organism and k (λ) is the extinction coefficient of the organism at the wavelength λ. The extinction coefficient of the organism represents absorption of the light into the organism.

Consequently, the transmittance is given by the following Equation (7):

$$t ( λ ) = exp ( - \frac{4 π k ( λ )}{λ} d ) \qquad ( 7 )$$

Next, the reflectance will be described. The reflectance should be calculated as the average of reflectances in all directions. In this example, only the reflectance to perpendicularly incident light will be considered for the sake of simplicity. Supposing the refractive index of the air is one, the reflectance to the perpendicularly incident light is given by the following Equation (8):

$$r ( λ ) = \frac{{( n ( λ ) - 1 )}^{2} + {k}^{2} ( λ )}{{( n ( λ ) + 1 )}^{2} + {k}^{2} ( λ )} \qquad ( 8 )$$

where n (λ) is the refractive index of the organism at the wavelength λ.

Consequently, the emissivity is given by the following Equation (9):

$$ɛ ( λ ) = 1 - r ( λ ) - t ( λ ) = 1 - \frac{{( n ( λ ) - 1 )}^{2} + {k ( λ )}^{2}}{{( n ( λ ) + 1 )}^{2} + {k ( λ )}^{2}} - exp ( - \frac{4 π k ( λ )}{λ} d ) \qquad ( 9 )$$

If the concentration of a constituent varies in an organism, the refractive index and the extinction coefficient of the organism will also change. The reflectance is usually as low as about 0.03 in the infrared range. Also, as can be seen from Equation (8), the reflectance does not depend on the refractive index or the extinction coefficient so much. That is why even if the refractive index and the extinction coefficient change due to a variation in biological constituent concentration, the reflectance will vary a little.

On the other hand, the transmittance heavily depends on the extinction coefficient as can be seen from Equation (7). For that reason, if the extinction coefficient of an organism (i.e., the degree of absorption of light into the organism) changes due to a variation in biological constituent concentration, the transmittance will change, too.

Thus, it can be seen that the radiation energy of the infrared radiation emitted as a thermal radiation from an organism depends on the concentration of the biological constituent. That is to say, the biological constituent concentration can be calculated based on the intensity of the radiation energy of the infrared radiation that has been emitted as a thermal radiation from the organism.

According to Equation (7), the transmittance depends on the thickness of the vital tissue. That is to say, the smaller the thickness of the vital tissue, the more significantly the transmittance will change with a variation in the extinction coefficient of the organism and the more easily the variation in biological constituent concentration can be detected.

The eardrum has such a small thickness of about 60 μm to about 100 μm as to be suitable for determining the biological constituent concentration using infrared radiation.

Hereinafter, first and second preferred embodiments of a measuring device according to the present invention will be described with reference to the accompanying drawings.

### EMBODIMENT 1

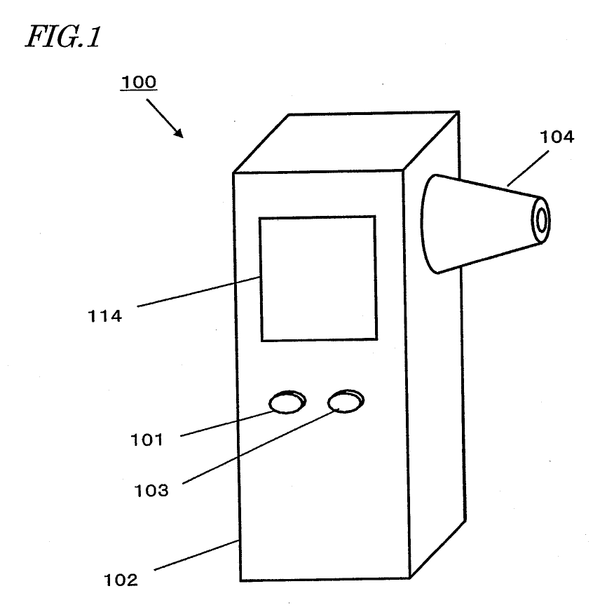

FIG. 1 is a perspective view illustrating the appearance of a biological constituent concentration measuring device 100 as a first specific preferred embodiment of the present invention.

The biological constituent concentration measuring device 100 (which will be simply referred to herein as a “measuring device 100”) includes a body 102 and a waveguide 104 arranged on a side surface of the body 102. The body 102 includes a display 114 to show the biological constituent concentration measured, a switch 101 to turn ON and OFF the measuring device 100, and another switch 103 to start the measuring process.

The measuring device 100 generates tilt information about a tilt angle of the eardrum based on a first piece of image capturing information obtained by capturing an image of a first area of the eardrum and a second piece of image capturing information obtained by capturing an image of a second area of the eardrum, which is different from the first area. Also, the measuring device 100 gets the infrared radiation, radiated from the eardrum, sensed by an infrared sensor, and calculates the concentration of the biological constituent based on the infrared radiation sensed and the tilt information. Then, the measuring device 100 outputs the information about the biological constituent concentration thus calculated onto the display 114, for example. As used herein, the “biological constituent concentration” is at least one of a glucose concentration (i.e., a blood glucose level), a hemoglobin concentration, a cholesterol concentration and a fat concentration.

The waveguide 104 is inserted into the acoustic foramen and has the function of guiding the infrared radiation, coming from the eardrum, into the measuring device 100. Anything may be used as the waveguide as long as it can guide infrared radiation. For example, a hollow tube or an optical fiber that transmits infrared radiation may be used. If a hollow tube is used, the inner surface of the hollow tube is preferably coated with a gold layer, which may be formed by either plating the inner surface of the hollow tube with gold or vapor-depositing gold on that surface.

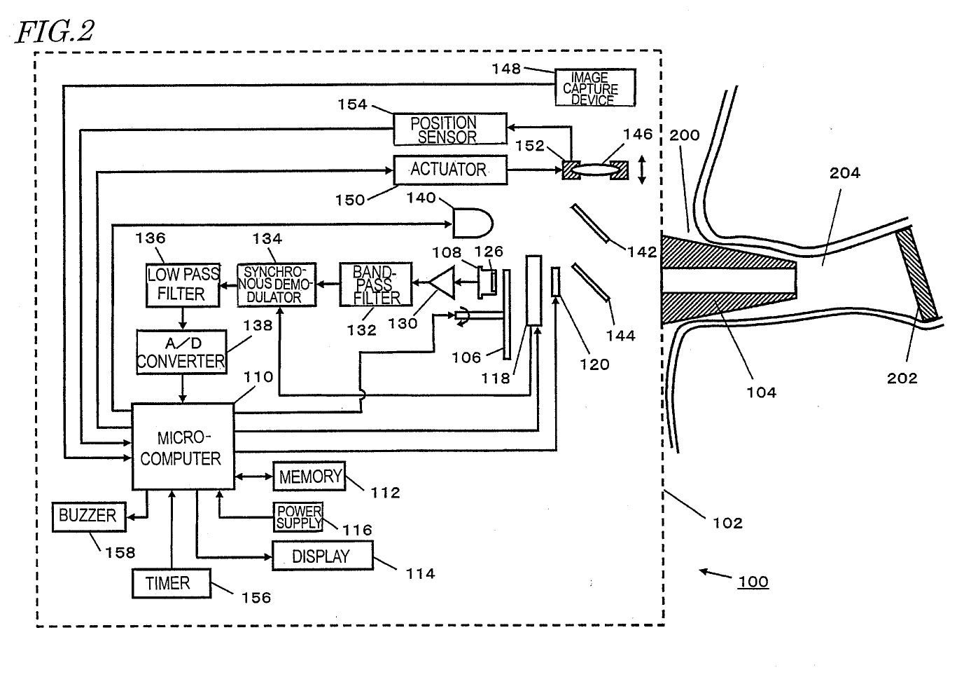

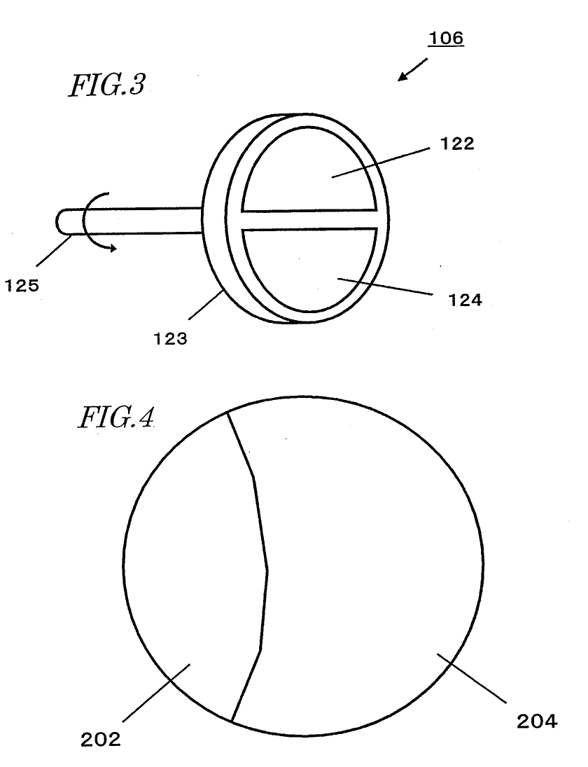

Next, the hardware configuration inside the body of the measuring device 100 will be described with reference to FIGS. 2 and 3.

FIG. 2 shows the hardware configuration of the measuring device 100.

The body of the measuring device 100 includes a chopper 118, a liquid crystal shutter 120, an optical filter wheel 106, an infrared sensor 108, a pre-amplifier 130, a band-pass filter 132, a synchronous demodulator 134, a low pass filter 136, an analog-to-digital (A/D) converter 138, a microcomputer 110, a memory 112, a display 114, a power supply 116, a light source 140, a first half mirror 142, a second half mirror 144, a condenser lens 146, an imager 148, an actuator 150, a lens frame 152, a position sensor 154, a timer 156 and a buzzer 158.

In the measuring device 100, the infrared sensor 108 detects the infrared radiation that has come from the eardrum. As used herein, the “infrared radiation coming from the eardrum” includes infrared radiation radiated from the eardrum as a thermal radiation from the eardrum itself and infrared radiation that has been radiated toward, and then reflected from, the eardrum. Unlike a measuring device according to the third preferred embodiment of the present invention to be described later, the measuring device 100 of this preferred embodiment has no light source that radiates infrared radiations. That is why the infrared sensor 108 of this preferred embodiment detects only the infrared radiation that has been radiated as a thermal radiation from the eardrum itself.

Any sensor may be used as the infrared sensor as long as the sensor can detect radiations having wavelengths falling within the infrared range of the spectrum. For example, the infrared sensor may be a pyroelectric sensor, a thermopile, a bolometer, an HgCdTe (MCT) detector or a Golay cell.

The microcomputer 110 may be a computer such as a central processing unit (CPU) or a digital signal processor (DSP). The microcomputer 110 has not only the function of generating information about the tilt angle of the eardrum based on the image information of the eardrum captured but also the function of calculating the biological constituent concentration with various factors caused by the tilt of the eardrum taken into consideration. The respective processes will be described later. The memory 112 functions as a storage device such as a RAM or a ROM.

The display 114 may be a liquid crystal display or an organic electroluminescent (EL) display, for example.

The power supply 116 provides AC or DC power to operate the electronic circuits inside the measuring device 100. A battery is preferably used as the power supply 116.

The chopper 118 chops the infrared radiation that has been radiated from the eardrum 202, guided into the body 102 through the waveguide 104 and then transmitted through the second half mirror 144, thereby transforming the infrared radiation into a high-frequency infrared signal. The operation of the chopper 118 is controlled in accordance with a control signal supplied from the microcomputer 110. The infrared radiation that has been chopped by the chopper 118 soon reaches the optical filter wheel 106.

FIG. 3 is a perspective view illustrating the optical filter wheel 106. The optical filter wheel 106 includes a first optical filter 122, a second optical filter 124, and a ring 127 to which these filters are fitted. The first and second optical filters 121 and 122 function as spectral filters. The wavelength ranges of infrared radiations to be transmitted through these filters will be described later.

In the example illustrated in FIG. 3, the first and second optical filters 122 and 124, both of which are semicircular, are fitted into the ring 123, thereby forming a disklike member. And at the center of that disklike member, arranged is a shaft 125. By rotating the shaft 125 in the direction pointed by the arrow shown in FIG. 3, the optical filters to pass the infrared radiation that has been chopped by the chopper 118 may be switched from one of the two optical filters 122 and 124 into the other.

The rotation of the shaft 125 is controlled by the microcomputer 110. The control signal supplied from the microcomputer 110 is sent to a motor (not shown), which spins the shaft 125 at a number of revolutions as defined by the control signal. The rotation of the shaft 125 is preferably controlled in accordance with a control signal supplied from the microcomputer 110. The shaft 125 preferably has its revolution synchronized with the rotation of the chopper 118 and is preferably controlled so as to turn 180 degrees while the chopper 118 is closed. This is because when the chopper 118 is opened next time, the infrared radiation to be chopped by the chopper 118 may be transmitted through the next optical filter.

These optical filters may be made by any known technique, which is not particularly limited herein but may be a vacuum evaporation process, for example. Specifically, the optical filters may be fabricated by stacking ZnS, MgF₂, PbTe, Ge, ZnSe and/or other layers on a substrate of Si, Ge or ZnSe by a vacuum evaporation process or an ion sputtering process, for instance.

In this case, an optical filter with a desired wavelength characteristic can be made by controlling the interference of light in a stack of thin-films with the thicknesses of the respective layers and the order or the number of times those layers are stacked on the substrate adjusted.

The infrared radiation that has been transmitted through the first or second optical filter 121 or 124 reaches the infrared sensor 108 with a sensing area 126. On reaching the infrared sensor 108, the infrared radiation is incident on the sensing area 126. The infrared sensor 108 receives the infrared radiation and transforms the infrared radiation into an electrical signal representing its intensity.

The electrical signal is output from the infrared sensor 108 to the pre-amplifier 130 and then amplified there. Then, the amplified electrical signal has its signal components filtered out by the band-pass filter 132 except those falling within a frequency range, of which the center frequency is defined by the chopping frequency. As a result, noise caused by some statistical fluctuation such as thermal noise can be minimized.

The electrical signal that has been subjected to the filtering process by the band-pass filter 132 is synchronized with the chopping frequency of the chopper 118 and integrated by the synchronous demodulator 134 so as to be demodulated into a DC signal.

Next, the electrical signal that has been demodulated by the synchronous demodulator 134 has its low frequency components filtered out by the low pass filter 136. In this manner, its noise can be further reduced.

Subsequently, the electrical signal that has been subjected to the filtering process by the low pass filter 136 is converted by the A/D converter 138 into a digital signal, which is then input to the microcomputer 110. In this case, the electrical signal that has come from any of the optical filters by way of the infrared sensor 108 can have its source identified (i.e., it is possible to determine which of those optical filters the infrared radiation, represented by the electrical signal, has been transmitted through) by using a control signal for the shaft 125 as a trigger. The duration of an electrical signal associated with the same optical filter is defined as an interval after the microcomputer has output a control signal for the shaft 125 and before it outputs the next shaft control signal. By calculating the integral of the electrical signals associated with the respective optical filters on the memory 112 and then working out its average, the noise can be further reduced. That is why the measured values are preferably integrated.

In the memory 112, stored is concentration correlation data that shows a correlation between the signal values of the electrical signals corresponding to the respective intensities of the infrared radiations transmitted through the first and second optical filters 122 and 124 and the biological constituent concentration. The microcomputer 110 reads this concentration correlation data from the memory 112, calculates a digital signal per unit time based on the digital signal that has been stored in the memory 112 by reference to the concentration correlation data, and converts the digital signal into a biological constituent concentration.

Then, the biological constituent concentration that has been worked out by the microcomputer 110 is output to, and presented on, the display 114.

The first optical filter 122 has such a spectral characteristic as to transmit infrared radiation that falls within a wavelength range including the wavelength to be absorbed into the biological constituent under measurement (which will be referred to herein as “measuring wavelength range”).

On the other hand, the second optical filter 124 has a different spectral characteristic from the first optical filter's 122. Specifically, the second optical filter 124 has such a spectral characteristic as to transmit infrared radiation that falls within a wavelength range including a wavelength to be absorbed into not the biological constituent under measurement but another biological constituent that would interfere with the measurement of the target biological constituent (which will be referred to herein as “reference wavelength range”). In this case, that another biological constituent may be any constituent that is included a lot in the organism other than the biological constituent under measurement.

For example, glucose has an infrared absorption spectrum with a peak of absorption in the vicinity of 9.6 μm. That is why if the biological constituent under measurement is glucose, the first optical filter 122 preferably has such a spectral characteristic as to transmit infrared radiation that falls within a wavelength range including 9.6 μm (e.g., 9.6±0.1 μm).

Meanwhile, protein, included a lot in an organism, would absorb infrared radiation around 8.5 μm, while glucose would not absorb infrared radiation around that wavelength. That is why the second optical filter 124 preferably has such a spectral characteristic as to transmit infrared radiation that falls within a wavelength range including 8.5 μm (e.g., 8.5±0.1 μm).

The concentration correlation data stored in the memory 112 to show the correlation between the respective signal values of the electrical signals representing the intensities of the infrared radiations that have been transmitted through the first and second optical filters 122 and 324 and the biological constituent concentration may be acquired in the following manner, for example.

First, as for a patient with a known biological constituent concentration such as a blood glucose level, the infrared radiation that has been emitted as a thermal radiation from his or her eardrum has its intensity measured. In this case, electrical signals representing the intensities of infrared radiations falling within the wavelength ranges to be transmitted by the first and second optical filters 122 and 124 are obtained. By making such measurement on a number of patients with mutually different biological constituent concentrations, multiple sets of data, each including the electrical signals representing the intensities of infrared radiations falling within the wavelength ranges to be transmitted by the first and second optical filters 122 and 124 and their associated biological constituent concentrations, can be collected.

Next, by analyzing these data sets that have been collected in this manner, concentration correlation data is obtained. For example, a multivariate analysis is carried out by either a multiple regression analysis such as partial least squares regression (PLS) method or a neural network method on the electrical signals representing the intensities of infrared radiations falling within the wavelength ranges to be transmitted by the first and second optical filters 122 and 124 and their associated biological constituent concentrations. As a result, a function showing a correlation between the electrical signals representing the intensities of infrared radiations falling within the wavelength ranges to be transmitted by the first and second optical filters 122 and 124 and their associated biological constituent concentrations can be obtained.

Also, the first optical filter 122 may have such a spectral characteristic as to transmit infrared radiation falling within a measuring wavelength range and the second optical filter 124 may have such a spectral characteristic as to transmit infrared radiation falling within a reference wavelength range. In that case, the difference between the signal values of the electrical signals representing the intensities of infrared radiations falling within the wavelength ranges to be transmitted by the first and second optical filters 122 and 342 may be calculated, and the correlation between that difference and its associated biological constituent concentration may be obtained as the concentration correlation data by performing a linear regression analysis such as a minimum square method, for example.

Next, the configuration for capturing an image of the eardrum 202 will be described.

The light source 140 emits visible radiation to illuminate the eardrum 202. The visible radiation that has been emitted from the light source 140 is reflected by the first half mirror 142 and by the second half mirror 144 and then guided through the waveguide 104 into an ear canal 204 to illuminate the eardrum 202.

As the light source 140, a visible radiation laser such as a red laser or a white LED may be used. Among other things, a white LED is preferred because the white LED energized generates a smaller quantity of heat than a halogen lamp and has less influence on the temperatures of the eardrum 202 and the ear canal 204.

The first half mirror 142 has the function of reflecting a part of visible radiation and transmitting the rest of it.

The second half mirror 144 reflects visible radiation and transmits infrared radiation. The second half mirror 144 is preferably made of a material that does not absorb but transmits infrared radiation and reflects visible radiation, e.g., ZnSe, CaF₂, Si or Ge. Alternatively, the second half mirror 144 may also have a structure in which an aluminum or gold layer with a thickness of several nanometers is deposited on a resin that is transparent to infrared radiation (such as polycarbonate).

Meanwhile, the visible radiation that has been reflected back from the eardrum 202 by way of the ear canal 204 and then entered the waveguide 104 is reflected by the second half mirror 144, but a part of the radiation is transmitted through the first half mirror 142. The visible radiation that has been transmitted through the first half mirror 142 is condensed by the condenser lens 146 held by the lens frame 152 to reach the imaging device 148. In this case, the condenser lens 146 is equivalent to the lens as defined by the claims of the present application.

As the imaging device 148, an imager such as a CMOS or a CCD may be used.

The measuring device 100 has a mechanism for converging the light on the imaging device 148 just as intended by detecting the distance from the imaging device 148 to the eardrum 202 and by driving the condenser lens 146 that is held on the lens frame 152.

In response to a control signal supplied from the microcomputer 110, the actuator 150 is driven so as to move the condenser lens 146 in the optical axis directions (i.e., in the directions pointed by the arrows in FIG. 2). In this case, the position of the condenser lens 146 is detected by a position sensor 154, which provides that information for the microcomputer 110.

Meanwhile, the microcomputer 110 gets high-frequency components extracted by a band-pass filter from the output signal of a pixel, which is included in the in-focus area around the center of the imaging device 148, and detects the contrast ratio according to the magnitude of the components extracted. And the microcomputer 110 controls the actuator 150 such that the condenser lens 146 moves to such a position that maximizes the contrast ratio.

Thus, even if the distance to the eardrum 202 has varied, an optical image of the eardrum 202 can also be formed properly on the imaging device 148. This mechanism does not measure the distance to the eardrum 202 directly but could be regarded as measuring the distance to the eardrum 202 indirectly based on the information about the position of the condenser lens 146.

The actuator 150 and the position sensor 154 may be identical with the ones used in an autofocusing mechanism for a known camcorder or digital still camera.

For example, the actuator 150 may include a coil attached to the lens frame 152, a yoke secured to the body 102, and a drive magnet attached to the yoke. The lens frame 152 may be supported on two guide poles so as to be movable in the optical axis directions. In that case, when current is supplied to the coil attached to the lens frame 152, magnetic driving force that drives the coil in the optical axis directions is generated in the coil located in a magnetic circuit that is formed by the yoke and the drive magnet. As a result, the lens frame 152 moves in the optical axis directions. The direction of the driving force may be controlled so as to be either positive or negative by changing the directions of the current supplied to the coil.

The position sensor 154 may include a sensor magnet, which is magnetized at a certain pitch and attached to the lens frame 152, and a magnetoresistance sensor (which will be referred to herein as an “MR sensor”) secured to the body 102, for example. By making the MR sensor secured to the body 102 detect the position of the sensor magnet attached to the lens frame 152, the position of the condenser lens 146 can be detected.

Hereinafter, a method for locating the eardrum 202 on the image that has been shot with the imaging device 148 will be described.

FIG. 4 illustrates an image of the acoustic foramen 200 that has been shot with the imaging device 148. A left-hand-side portion of the image represents the eardrum 202, while a right-hand-side portion thereof represents the ear canal 204. The position at which the eardrum 202 can be recognized and the size of the eardrum 202 will change not only from one person to another but also with how deep the waveguide 104 has been inserted.

The ear canal looks flesh-colored, while the eardrum looks either white or uncolored and transparent. If the image capturing information detecting section can sense this difference in color between the ear canal and the eardrum, they can be distinguished from each other. And by getting the image information, which has been obtained by the imaging device 148, subjected to image processing by the microcomputer 110, the area representing the eardrum 202 is extracted from the image information. As the image processing method, an area extracting technique that adopts threshold value processing and labeling processing in combination may be used as will be described below.

First, the microcomputer 110 performs the threshold value processing on the image information. Each pixel of an image has values representing the colors red (R), green (G) and blue (B), which will be referred to herein as “RGB values”. And the average of these RGB values represents the brightness of each pixel.

By setting a predetermined reference value (i.e., a threshold value) with respect to the brightnesses of respective pixels, the microcomputer 110 performs the processing of converting the brightnesses of respective pixels into the two values representing black and white, respectively, by reference to the threshold value. Suppose the threshold value is determined in advance when the measuring device 100 is shipped, for example. In that case, if the brightness of a given pixel is equal to or greater than the threshold value, the microcomputer 110 regards the pixel as representing white. Otherwise, the microcomputer 110 regards the pixel as representing black. In general, the pixels in the area representing the eardrum 202 are brighter than the pixels in the area representing the ear canal 204. That is why if the threshold value is set between the brightnesses of pixels in the area representing the eardrum and those of pixels in the area representing the ear canal, then the pixels in that area representing the eardrum 202 will be white and the ones in the area representing the ear canal 204 will be black as a result of the processing described above.

Next, the microcomputer 110 performs labeling processing on the image information that has been subjected to the threshold value processing described above. For example, the microcomputer 110 may scan all pixels included in the image information that has been subjected to the threshold value processing and may attach the same label to all pixels representing the color white as their attribute value.

By performing the processing described above, the microcomputer 110 can recognize the area, consisting of those labeled pixels, as representing the eardrum 202. The ratio of the area representing the eardrum 202 to the entire image captured can be calculated by the microcomputer 110 as the ratio of the number of those labeled pixels to the number of all pixels.

The liquid crystal shutter 120 has a structure in which a number of liquid crystal cells are arranged in a matrix pattern. By changing voltages applied to the respective liquid crystal cells, those cells can be controlled independently of each other so as to have either a light transmitting state or a light cutoff state. The liquid crystal shutter may include TFTs (thin-film transistors), for example, and is preferably able to control the transmission and cutoff of light using the TFTs.

On recognizing, as a result of the image processing described above, an image portion representing the eardrum 202 in the image information that has been captured with the imaging device 148, the microcomputer 110 controls the voltages to be applied to the respective liquid crystal cells of the liquid crystal shutter 120, thereby changing the states of liquid crystal cells, on which infrared radiation that has come from the eardrum 202 is going to be incident, into the light-transmitting state, and changing the states of other liquid crystal cells, on which infrared radiation that has come from elsewhere is going to be incident, into the light-cutoff state.

By using the liquid crystal shutter 120 as an optical path control element in this manner, the infrared radiation that has been radiated from the eardrum will reach the infrared sensor 108 but the infrared radiation that has been radiated from the ear canal will be cut off and never reach the infrared sensor 108. Thus, the influence of such infrared radiation that has come from the ear canal can be eliminated. As a result, the measuring can get done even more accurately.

Optionally, a mechanical shutter may also be used as an alternative optical path control element instead of such a liquid crystal shutter. As the mechanical shutter, a known digital mirror device (which will be abbreviated herein as “DMD”), in which a number of micro mirrors are arranged so as to form a plane by the MEMS technology, may be used, for example. The DMD may be fabricated by a known MEMS (microelectromechanical system) technology. Each of those micro mirrors can be controlled so as to be turned ON or OFF by driving an electrode arranged under the mirror surface. Specifically, when turned ON, the micro mirror reflects the infrared radiation, which has been radiated from the eardrum, toward the infrared sensor. On the other hand, when turned OFF, the micro mirror reflects the incoming infrared radiation toward an absorbing member, which is arranged inside the DMD, not toward the infrared sensor. That is why by driving the respective micro mirrors independently of each other, the projection of the infrared radiation can be controlled on a micro area basis.

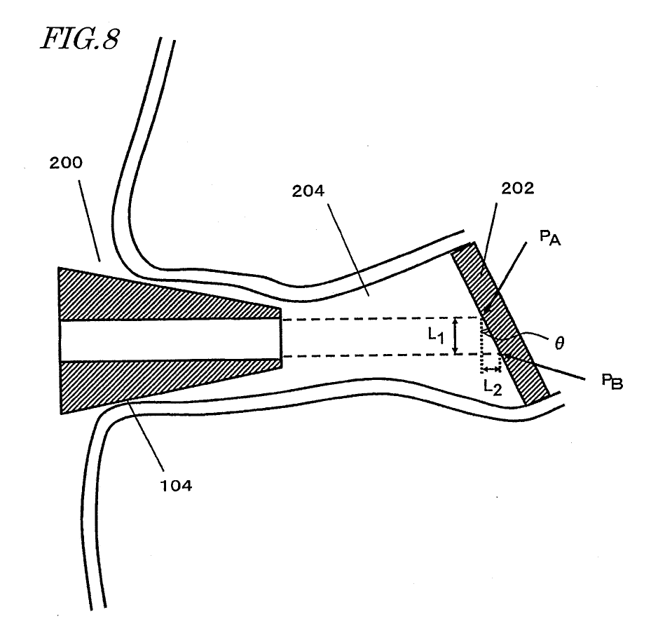

Next, a method for estimating the tilt angle defined by the eardrum 202 with respect to the infrared radiation incident plane of the infrared sensor 108 using an image that has been shot with the imaging device 148 will be described with reference to FIGS. 5 through 8. Specifically, FIGS. 5, 6 and 7 illustrate the states of pixels representing the eardrum 202 on the image that has been shot with the imaging device 148. For convenience sake, this image shot is supposed to consist of only the portion representing the eardrum. However, in a situation where the image shot includes a portion representing the eardrum 202 and a portion representing the ear canal 204 as shown in FIG. 4, the same processing may be carried out on only the image portion representing the eardrum 202. FIG. 8 is a cross-sectional view showing where the waveguide 104 that has been inserted into the acoustic foramen 200 is located with respect to the eardrum 202.

The microcomputer 110 gets high-frequency components extracted by a band-pass filter from the output signals of pixels, which have been determined by the method described above to be located within the area representing the eardrum 202 among all pixels of the imaging device 148, and detects the contrast ratios based on the magnitudes of those components extracted. Then, the microcomputer 110 compares the contrast ratios to the threshold value, thereby regarding pixels with contrast ratios that are equal to or greater than the threshold value as in-focus pixels.

FIG. 5 illustrates an image of the eardrum 202 that has been shot with the imaging device 148 when the condenser lens 146 is located at a first position. Among the multiple pixels 501 that are arranged in a matrix pattern, the black ones around the top left corner are a group of pixels 502 in the in-focus area, while the white ones are a group of pixels 503 in the out-of-focus area. If one side of the eardrum 202 that faces the ear canal 204 is approximated as a plane, the group of pixels 502 in the in-focus area will be arranged in line on the image that has been shot with the imaging device 148.

Next, the microcomputer 110 controls the actuator 150 to move the condenser lens 146. In the example to be described below, the condenser lens 146 is supposed to move from the first position toward the imaging device 148 and reach a second position.

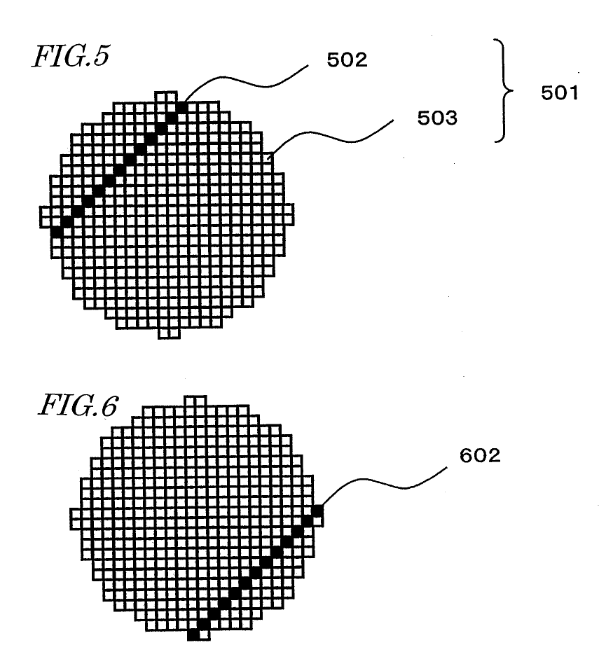

FIG. 6 illustrates an image of the eardrum 202 that has been shot with the imaging device 148 when the condenser lens 146 is located at the second position. As the condenser lens 146 has moved from the first position to the second position, the condenser lens 146 comes to have a longer focal length, and the focal point is formed at a longer distance beyond the eardrum 202 compared to the image shown in FIG. 5. A group of pixels 602 in the in-focus area is shown in FIG. 6. Compared to the group of pixels 502 shown in FIG. 5, the group of pixels 602 has moved toward the bottom right corner of the paper.

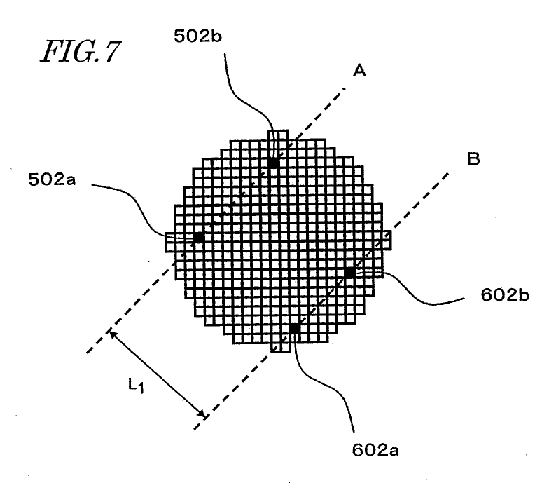

FIG. 7 shows the group of linearly arranged pixels A (which will be referred to herein as “pixels in line A”) when the condenser lens 146 is located at the first position and the group of linearly arranged pixels B (which will be referred to herein as “pixels in line B”) when the condenser lens 146 is located at the second position. As shown in FIG. 7, the microcomputer 110 extracts at least two pixels 502a and 502b from the group of in-focus pixels 502 when the condenser lens 146 is located at the first position and further extracts at least two more pixels 602a and 602b from the group of in-focus pixels 602 when the condenser lens 146 is located at the second position. Then, the microcomputer 110 calculates the interval L1 between the line A that connects together the two extracted pixels 502a and 502b and the line B that connects together the two extracted pixels 602a and 602b.

In the cross section of the eardrum 202 shown in FIG. 8, the location corresponding to the line A is identified by P_{A} and the location corresponding to the line B is identified by P_{B}. Also, in FIG. 8, the interval L2 corresponds to the difference between the focal length when the condenser lens 146 is located at the first position and the focal length when the condenser lens 146 is located at the second position. That is to say, the interval L2 is equal to the distance that the condenser lens 146 should go when the microcomputer 110 controls the actuator 150 such that the condenser lens 146 moves from the first position to the second position.

In the preferred embodiment described above, the distance the condenser lens 146 needs to go is determined using the position sensor. However, the distance to go for the condenser lens 146 could also be determined even without the position sensor. For example, if the position can be determined by the value of the voltage applied to the actuator 316, then the distance to go can be calculated based on the difference between the voltage values associated with the first and second positions of the lens. Alternatively, if the relation between the variation in the voltage applied to the actuator 316 and the distance to go is known, then the distance can also be determined by the variation in the voltage applied to move the lens from the first position to the second position.

Also, in the example illustrated in FIGS. 5 and 6, two pixels are extracted from each of the two groups of pixels 502 and 602 representing the eardrum 202 to draw the lines A and B and calculate the interval L₁ between those two lines A and B. However, this processing does not always require the use of multiple pixels. Thus, the interval L₁ can also be calculated even if just one pixel is extracted from one or both of these two groups. For example, if there is only one pixel representing the eardrum 202 when the condenser lens 146 is located at the first position but if there are multiple pixels representing the eardrum 202 when the condenser lens 146 is located at the second position, then the interval between the point and the line may be calculated. Also, if only one pixel represents the eardrum 202 in both of these two situations, then the interval L₁ may be obtained as the length of the line segment that connects those two points together.

As can be seen from FIG. 2, the infrared radiation incident plane of the infrared sensor 108 is parallel to the end face of the waveguide 104 that has been inserted into the acoustic foramen 200. That is why in this example, instead of estimating the tilt angle of the eardrum 202 with respect to the infrared radiation incident plane of the infrared sensor 108, the tilt angle defined by the eardrum 202 with respect to the end face of the waveguide 104 that has been inserted into the acoustic foramen 200 is estimated.

The interval L₂ described above can be determined arbitrarily. For that reason, if the interval L₂ is fixed at a predetermined value, the tilt angle of the eardrum 202 can be estimated just by calculating the interval L₁. And the interval L₁ is calculated based on the outputs of two pixels that are associated with two imaging points when the condenser lens 146 is located at the first and second positions, respectively. Consequently, information about the tilt angle of the eardrum can be obtained from only the image capturing information.

As shown in FIG. 8, the tilt angle defined by the eardrum 202 with respect to the end face of the waveguide 104 that has been inserted into the acoustic foramen 200 can be represented by the ratio of the interval L₂ to the interval L₁. For example, supposing the tilt angle defined by the eardrum 202 with respect to the end face of the waveguide 104 is identified by θ (see FIG. 8), tan θ=L₂/L₁ is satisfied. That is why by calculating the intervals L₁ and L₂ using the microcomputer 110, the tilt angle defined by the eardrum 202 with respect to the end face of the waveguide 104 that has been inserted into the acoustic foramen 200 can be estimated.

Hereinafter, it will be described how this measuring device 100 works. In the following description, the user of the measuring device 100 is supposed to measure the concentration of his or her own biological constituent. The same statement will apply to the second and third preferred embodiments of the present invention to be described later.

First, when the user presses the power switch 101 of the measuring device 100, the power is turned ON inside the body 102 to get the measuring device 100 ready to make measurements.

Next, the user holds the body 102 in his or her hand to insert the waveguide 104 into his or her acoustic foramen 200. The waveguide 104 is a conical hollow tube that increases its diameter from the end of the waveguide 104 toward the portion connected to the body 102. That is why the waveguide 104 has such a structure as to prevent itself from being inserted any deeper than the position where the outside diameter of the waveguide 104 gets equal to the inside diameter of the acoustic foramen 200.

Subsequently, when the user who is holding the measuring device 100 presses the measuring start switch 103 of the measuring device 100 at the position where the outside diameter of the waveguide 104 gets equal to the inside diameter of the acoustic foramen 200, the light source 140 inside the body 102 is turned ON and the imaging device 148 start capturing an image.

Next, the processing of locating the eardrum 202 by the method described above on the image that has been captured with the imaging device 148 is performed. If as a result of the imaging, the microcomputer 110 has determined that there is no image portion representing the eardrum 202 on the image that has been shot with the imaging device 148, then a message notifying the user that the waveguide 104 inserted is misaligned with the eardrum 202 is put on the display 114, the buzzer 158 is activated, and/or a voice message or an alarm is output through a loudspeaker (not shown), thereby giving the user an alert and notifying him or her of the error. In this case, the user may be notified of the error only if the ratio of the eardrum area to the entire image shot, which has been calculated by the microcomputer 110, is equal to or smaller than a threshold value. Even if the user receives such an error message telling that the eardrum 202 couldn't be located, he or she just needs to adjust the inserting direction of the waveguide 104 by moving the measuring device 100.

On the other hand, if as a result of the imaging, the microcomputer 110 has determined that the eardrum 202 has been located successfully on the image that has been shot with the imaging device 148, then the microcomputer 110 calculates the intervals L₁ and L₂ by the method described above and estimates the tilt angle of the eardrum with respect to the end face of the waveguide 104 that has been inserted into the acoustic foramen 200.

Also, on determining that the eardrum 202 has been located successfully on the image that has been shot with the imaging device 148 and that the tilt angle of the eardrum 202 has been estimated, the microcomputer 110 shows a message telling that the eardrum 202 has been located on the display 114, makes the buzzer 158 beep or outputs a voice message or an alarm through a loudspeaker (not shown), thereby notifying the user of that.

Once the eardrum 202 has been located successfully, the infrared radiation radiated from the eardrum 202 starts to be measured automatically. By notifying the user that the eardrum 202 has been located successfully, he or she can know that the measuring process has started and that he or she just needs to keep the measuring device 100 in place without moving it.

Also, on determining that the eardrum 202 has been located successfully on the image that has been shot with the imaging device 148, the microcomputer 110 controls the voltages to be applied to the respective liquid crystal cells of the liquid crystal shutter 120, thereby changing the states of the liquid crystal cells, on which the infrared radiation that has come from the eardrum 202 is going to be incident, into the light transmitting state and those of the liquid crystal cells, on which infrared radiation that has come from elsewhere is going to be incident, into the light cutoff state. Furthermore, the microcomputer 110 activates the chopper 118, thereby starting to measure the infrared radiation that has been radiated from the eardrum 202.

Even after the infrared radiation has started to be measured, the processing of locating the eardrum on the image that has been shot with the imaging device 148 is still performed continuously. If during the measurements, the user has happened to remove the waveguide 104 from the acoustic foramen 200 accidentally or change the directions of the waveguide 104 significantly, then the microcomputer 110 determines that there is no image portion representing the eardrum 202 on the image that has been shot with the imaging device 148, thereby sensing the user's mistake. On sensing such a mistake, the microcomputer 110 may show a message telling that the waveguide 104 inserted is misaligned with the eardrum 202 on the display 114, make the buzzer 158 beep, and/or output a voice message or an alarm through a loudspeaker (not shown), thereby notifying him or her of that mistake. Furthermore, the microcomputer 110 controls the chopper 118, thereby cutting off the infrared radiation that is going to reach the optical filter wheel 106 and stopping the measurements automatically.

In this case, the user may be notified of (or alerted to) the error if the ratio of the eardrum area to the entire image shot, which has been calculated by the microcomputer 110, is equal to or smaller than a threshold value. Even if the user receives such an error message telling that the eardrum 202 couldn't be located, he or she just needs to insert the waveguide 104 into the acoustic foramen 200 again or adjust the inserting direction of the waveguide 104 by moving the measuring device 100 and then press the measuring start switch 103. Then, the measuring process gets started again.

Still alternatively, the measuring device 100 may notify the user with the frequencies or intensities of the sound changed according to the area ratio of the eardrum area to the entire image shot.

On sensing, by reference to the clock signal supplied from the timer 156, that a predetermined amount of time has passed since the measuring process was started, the microcomputer 110 controls the chopper 118 to block the infrared radiation from reaching the optical filter wheel 106. As a result, the measuring process ends automatically. At this point in time, by controlling the display 114 or the buzzer 158, the microcomputer 110 shows a message telling that the measuring process has ended on the display 114, makes the buzzer 158 beep or outputs a voice message or an alarm through the loudspeaker (not shown), thereby notifying the user of the end of the measuring process. On confirming that the measuring process has ended, the user can now remove the waveguide 104 from his or her acoustic foramen 200.

The electrical signal supplied from the A/D converter 138 is corrected by the microcomputer 110 based on the area ratio of the eardrum area to the entire image shot and on the tilt angle of the eardrum with respect to the end face of the waveguide 104 that has been inserted into the acoustic foramen 200, both of which have been obtained by the methods described above.

A specific method of correcting the electrical signal based on the area ratio of the eardrum area to the entire image captured may be selected according to the contents of the electrical signal represented by the correlation data that is stored in the memory 112. For example, if the electrical signal represented by the correlation data that is stored in the memory 112 is a signal generated per unit area, then the electrical signal measured may be corrected into such a signal generated per unit area by using the area ratio of the eardrum area to the entire image shot. In this manner, the measured signal can be corrected with the area of the eardrum that has been irradiated with the infrared radiation measured.

The intensity of the infrared radiation radiated from an organism depends on the area of the portion through which the infrared radiation is radiated. That is why even if the area of the eardrum that has been captured with the imaging device has varied, that variation in the results of measurements can be reduced by making the corrections described above and the measurements can get done even more accurately.

Meanwhile, the correction of the electrical signal based on the tilt angle defined by the eardrum with respect to the end face of the waveguide 104 that has been inserted into the acoustic foramen 200 can be made by dividing the electrical signal S₀ measured by cos θ as can be seen from FIG. 8. That is why the corrected electrical signal S can be calculated by the following Equation (10) using the intervals L₁ and L₂:

$$S = \frac{{S}_{0}}{cos θ} = \frac{{S}_{0} \sqrt{{L}_{1}^{2} + {L}_{2}^{2 }}}{{L}_{1}} \qquad ( 10 )$$

The microcomputer 110 reads concentration correlation data, representing a correlation between the electrical signals representing the respective intensities of the infrared radiations that have been transmitted through the first and second optical filters 122 and 124 and the concentration of the biological constituent, from the memory and converts the corrected electrical signals into biological constituent concentrations by reference to the concentration correlation data. The biological constituent concentrations thus obtained are shown on the display 114.

As described above, the measuring device 100 of this preferred embodiment corrects the generated signal with the tilt angle of the eardrum with respect to the end face of the waveguide 104 that has been inserted into the acoustic foramen (i.e., the tilt angle defined by the eardrum with respect to the infrared radiation incident plane of the infrared sensor 108). Thus, it is possible to reduce the influence of individual differences in the angle defined by the eardrum with respect to a plane that intersects at right angles with an axis that connects together the center of the entrance of the ear canal and the navel of the eardrum. And the influence of the variation in the angle of insertion of the waveguide 104 into the acoustic foramen 200 can also be reduced. As a result, the concentration of the biological constituent can be measured highly accurately.

### Embodiment 2

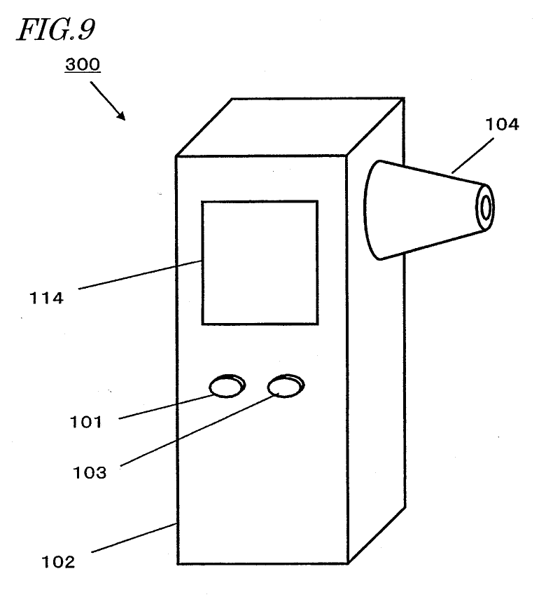

FIG. 9 is a perspective view illustrating the appearance of a biological constituent concentration measuring device 300 (which will be simply referred to herein as an “measuring device 300”) according to a second specific preferred embodiment of the present invention. The biological constituent concentration measuring device 300 includes a body 102 and a waveguide 104 arranged on a side surface of the body 102. The body 102 includes a display 114 to show the biological constituent concentration measured, a switch 101 to turn ON and OFF the measuring device 100, and another switch 103 to start the measuring process.

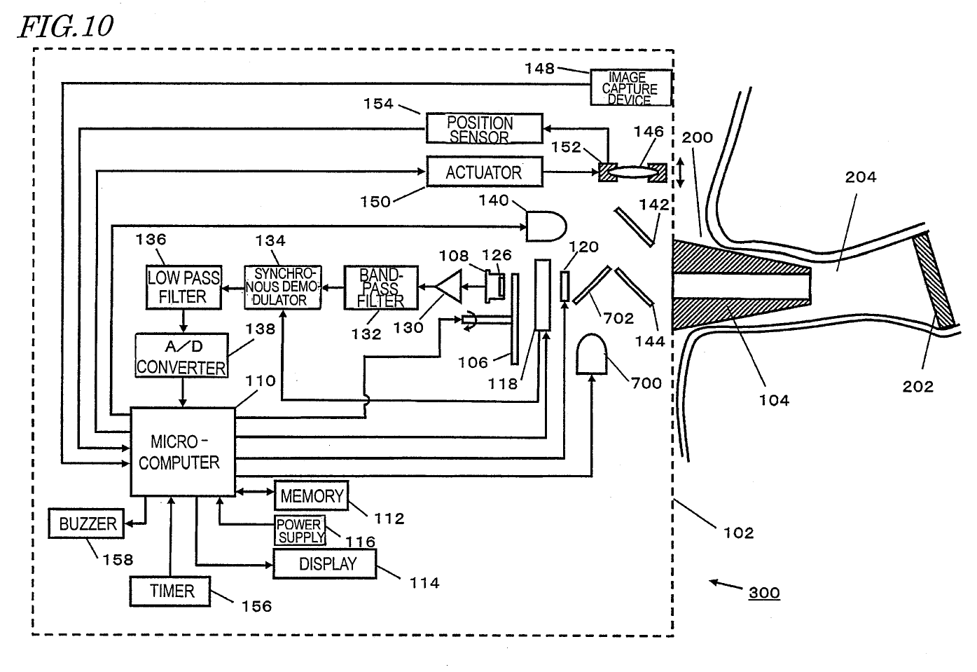

Hereinafter, the internal configuration of the body of the measuring device 300 of this preferred embodiment will be described with reference to FIG. 10, which shows the hardware configuration of the measuring device 300 of this preferred embodiment.

Unlike the measuring device 100 of the first preferred embodiment described above, the body of the measuring device 300 includes an infrared radiation source 700 for emitting infrared radiation and a half mirror 702. In the other respects, the measuring device 300 has quite the same configuration as the measuring device 100 of the first preferred embodiment described above, and the description thereof will be omitted herein.

The infrared radiation source 700 emits infrared radiation to irradiate the eardrum 202. The infrared radiation that has been emitted from the infrared radiation source 700, reflected by the third half mirror 702 and then transmitted through the second half mirror 144 is guided through the waveguide 104 to enter the ear canal 204 and irradiate the eardrum 202. The infrared radiation that has reached the eardrum 202 is reflected from the eardrum 202 back toward the measuring device 300 as reflected light. This infrared radiation is guided through the waveguide 104, transmitted through the second and third half mirrors 144 and 702 and the optical filter wheel 106, and then detected at the infrared sensor 108.

In this preferred embodiment, the intensity of the light reflected from the eardrum 202 and then detected is calculated as the product of the reflectance given by Equation (8) and the intensity of the infrared radiation impinging on the eardrum 202. As can be seen from Equation (8), as the biological constituent changes its concentrations, the refractive index and the extinction coefficient of the organism change. The reflectance is normally as small as about 0.03 in the infrared range of the spectrum and depends very little on the refractive index and the extinction coefficient as can be seen from Equation (8). The reflectance hardly varies even when the biological constituent changes its concentrations. However, if the intensity of the infrared radiation emitted from the infrared radiation source 700 is increased, the variation in reflectance can be sensed.

As the infrared radiation source 700, any known light source may be used without restriction. For example, a silicon carbide light source, a ceramic light source, an infrared LED, or a quantum cascade laser may be used.

The third half mirror 702 has the function of splitting infrared radiation into two bundles of rays. The third half mirror 702 may be made of ZnSe, CaF₂, Si, Ge or any other suitable material. Furthermore, to control the transmittance and reflectance of the infrared radiation, the third half mirror is preferably coated with an antireflection film.

The concentration correlation data stored in the memory 112 to show the correlation between the respective signal values of the electrical signals representing the intensities of the infrared radiations that have been transmitted through the first and second optical filters 122 and 324 and the biological constituent concentration may be acquired in the following manner, for example.

First, as for a patient with a known biological constituent concentration such as a blood glucose level, the infrared radiation that has been emitted from the infrared radiation source 700 toward, and then reflected from, his or her eardrum has its intensity measured. In this case, electrical signals representing the intensities of infrared radiations falling within the wavelength ranges to be transmitted by the first and second optical filters 122 and 124 are obtained. By making such measurement on a number of patients with mutually different biological constituent concentrations, multiple sets of data, each including the electrical signals representing the intensities of infrared radiations falling within the wavelength ranges to be transmitted by the first and second optical filters 122 and 124 and their associated biological constituent concentrations can be collected.

Next, by analyzing these data sets that have been collected in this manner, concentration correlation data is obtained. For example, a multivariate analysis is carried out by either a multiple regression analysis such as partial least squares regression (PLS) method or a neural network method on the electrical signals representing the intensities of infrared radiations falling within the wavelength ranges to be transmitted by the first and second optical filters 122 and 124 and their associated biological constituent concentrations. As a result, a function showing a correlation between the electrical signals representing the intensities of infrared radiations falling within the wavelength ranges to be transmitted by the first and second optical filters 122 and 124 and their associated biological constituent concentrations can be obtained.

By detecting the infrared radiation that has been emitted from the infrared radiation source 700 toward, and then reflected from, the eardrum, the biological constituent concentration can be measured.

Hereinafter, it will be described how the measuring device 300 of this preferred embodiment operates. The measuring device 400 operates in quite the same way as the measuring device 100 of the first preferred embodiment described above since its power has been turned ON and until the waveguide is inserted into the ear and the tilt angle of the eardrum 202 gets estimated, and the description thereof will be omitted herein.

Also, on determining that the eardrum 202 has been located successfully on the image that has been shot with the imaging device 148 and that the tilt angle of the eardrum 202 has been estimated, the microcomputer 110 shows a message telling that the eardrum 202 has been located on the display 114, makes the buzzer 158 beep, and/or outputs a voice message or an alarm through the loudspeaker (not shown), thereby notifying the user of that.

Once the eardrum 202 has been located successfully, infrared radiation is emitted from the infrared radiation source 700 automatically. And the infrared radiation is reflected from the eardrum 202, is radiated again from the eardrum 202, and then starts to be measured. By notifying the user that the eardrum 202 has been located successfully, he or she can know that the measuring process has started and that he or she just needs to keep the measuring device 100 in place without moving it.

Also, on determining that the eardrum 202 has been located successfully on the image that has been shot with the imaging device 148, the microcomputer 110 controls the voltages to be applied to the respective liquid crystal cells of the liquid crystal shutter 120, thereby changing the states of the liquid crystal cells, on which the infrared radiation that has come from the eardrum 202 is going to be incident, into the light transmitting state and those of the liquid crystal cells, on which infrared radiation that has come from elsewhere is going to be incident, into the light cutoff state. Furthermore, the microcomputer 110 activates the chopper 118, thereby starting to measure the infrared radiation that has been radiated from the eardrum 202.

Even after the infrared radiation has started to be measured, the processing of locating the eardrum on the image that has been shot with the imaging device 148 is still performed continuously. If during the measurements, the user has happened to remove the waveguide 104 from the acoustic foramen 200 accidentally or change the directions of the waveguide 104 significantly, then the measuring device 400 performs the same processing as the measuring device 100 of the first preferred embodiment described above.

On sensing, by reference to the clock signal supplied from the timer 156, that a predetermined amount of time has passed since the measuring process was started, the microcomputer 110 controls the infrared radiation source 700 to cut off the infrared radiation. As a result, the measuring process ends automatically. At this point in time, by controlling the display 114 or the buzzer 158, the microcomputer 110 shows a message telling that the measuring process has ended on the display 114, makes the buzzer 158 beep, or outputs a voice message or an alarm through a loudspeaker (not shown), thereby notifying the user of the end of the measuring process. On confirming that the measuring process has ended, the user removes the waveguide 104 from his or her acoustic foramen 200.

The method of correcting the electrical signal supplied from the A/D converter 138 is the same as the one adopted by the measuring device 100 of the first preferred embodiment described above. Likewise, the method of correcting an electrical signal with the tilt angle of the eardrum with respect to the end face of the waveguide 104 that has been inserted into the acoustic foramen 200 and the method of calculating the concentration of a biological constituent are also the same as what is used by the measuring device 100 of the first preferred embodiment. Thus, the description thereof will be omitted herein.

In the preferred embodiments described above, an optical filter wheel is supposed to be used as a spectral element. However, any other spectral element may be used as long as the element can split infrared radiation into multiple rays with mutually different wavelengths. For example, a Michelson interferometer or a diffraction grating for transmitting infrared radiation falling within a particular wavelength range may be used. Besides, there is no need to integrate a number of filters together as in the optical filter wheel. Furthermore, when an infrared radiation source such as an infrared LED or a quantum cascade laser that can emit a radiation with a particular wavelength is used, there is no need to split the infrared radiation. In that case, the first and second optical filters provided for the optical filter wheel of the preferred embodiment described above are no longer necessary.

As described above, the measuring device 300 of this preferred embodiment corrects the generated signal with the tilt angle of the eardrum with respect to the end face of the waveguide 104 that has been inserted into the acoustic foramen 200 (i.e., the tilt angle defined by the eardrum with respect to the infrared radiation incident plane of the infrared sensor 108). Thus, it is possible to reduce the influence of individual differences in the angle defined by the eardrum with respect to a plane that intersects at right angles with an axis that connects together the center of the entrance of the ear canal and the navel of the eardrum. And the influence of the variation in the angle of insertion of the waveguide 104 into the acoustic foramen 200 can also be reduced. As a result, the concentration of the biological constituent can be measured highly accurately.

### INDUSTRIAL APPLICABILITY

A biological constituent concentration measuring device according to the present invention can be used effectively to measure a biological constituent concentration non-invasively, e.g., measure a glucose concentration without collecting blood.

1. A measuring device for measuring concentration of a biological constituent, the measuring device comprising:
an image capturing section for capturing an image of an eardrum;
a processing section for generating tilt information concerning tilt of the eardrum based on a first image capturing information obtained by capturing an image of a first area of the eardrum and a second capturing information obtained by capturing an image of a second area of the eardrum, which is different from the first area;
an infrared sensor for sensing infrared radiation that has been radiated from the eardrum; and
a computing section for calculating concentration of a biological constituent based on the infrared radiation sensed and the tilt information.

2. The measuring device of claim 1, wherein the image capturing section includes an imaging device that has multiple pixels, and
wherein the processing section generates the tilt information by using an output of one of the multiple pixels that is associated with an imaging point in the first area as the first image capturing information and using an output of another one of the multiple pixels that is associated with an imaging point in the second area as the second image capturing information.

3. The measuring device of claim 2, wherein the image capturing section further includes:
a light source that emits light;
a lens for condensing the light, which has been emitted and then reflected from an acoustic foramen, onto the imaging device;
an actuator for driving the lens;
an actuator control section for controlling the actuator; and
an extracting section for extracting the output of one of the pixels that is associated with an in-focus area based on image capturing information that has been obtained by the imaging device, and
wherein the extracting section extracts, as the first image capturing information, the output of at least one first pixel that is associated with the first area in which the light is focused when the lens is located at a first position and the extracting section also extracts, as the second image capturing information, the output of at least one second pixel that is associated with the second area in which the light is focused when the lens is located at a second position, and
wherein the processing section calculates an interval between the first and second pixels based on the first image capturing information and the second image capturing information, and
wherein the computing section calculates the concentration of the biological constituent based on the interval and the infrared radiation sensed.

4. The measuring device of claim 3, wherein the processing section calculates a distance that the lens has gone when reaching the second position from the first position, and
wherein the computing section calculates the concentration of the biological constituent further based on the distance.

5. The measuring device of claim 3, further comprising:
a detecting section for detecting an image portion corresponding to the eardrum based on the image capturing information that has been provided as an image from the image capturing section, and
an optical path control element for controlling the optical path of the infrared radiation that has been radiated from the eardrum based on the image portion detected such that the infrared radiation is selectively incident on one of the multiple pixels, associated with the image portion, on the imaging device.

6. The measuring device of claim 3, further comprising a waveguide to be inserted into the acoustic foramen,
wherein the waveguide outputs to the acoustic foramen the light that has been emitted from the light source and receives the light that has been reflected from the acoustic foramen and the infrared radiation that has been radiated from the eardrum.

7. The measuring device of claim 1, further comprising an infrared radiation source for increasing intensity of the infrared radiation that has been radiated from the eardrum,
wherein the detecting section outputs a signal representing the intensity of the infrared radiation received.

8. The measuring device of claim 1, further comprising an output section for outputting information concerning the calculated concentration of the biological constituent.

9. The measuring device of claim 8, wherein the output section outputs the information about the biological constituent concentration to a display.
